(19) United States
(12) Patent Application Publication
(10) Pub. No.: US 2014/0271521 A1
(43) Pub. Date: Sep. 18, 2014

(54) Process of Forming a Personal Care Article

(71) Applicant: The Procter & Gamble Company, Cincinnati, OH (US)

(72) Inventors: Robert Wayne GLENN, JR., Liberty Twp, OH (US); Todd Ryan THOMPSON, Loveland, OH (US); Ungyeong JUNG, North Royalton, OH (US); Changwei ZHU, Richmond Hill (CA); Alireza TABATABAEI-NAEINI, Toronto (CA)

(73) Assignee: The Procter & Gamble Company, Cincinnati, OH (US)

(21) Appl. No.: 14/212,522

(22) Filed: Mar. 14, 2014

Related U.S. Application Data
(60) Provisional application No. 61/798,061, filed on Mar. 15, 2013.

(51) Int. Cl. A61K 8/46; A61K 8/898; A61K 8/73; A61Q 5/02; A61K 8/81; A61Q 19/10; A61Q 5/12; A61K 8/89; A61K 8/65

(52) U.S. Cl. CPC A61K 8/463; A61K 8/89; A61K 8/898; A61K 8/731; A61K 8/732; A61K 8/65; A61K 8/73; A61K 8/8176; A61K 8/8129; A61Q 19/10; A61Q 5/12; A61Q 5/02
USPC 424/70.122; 510/475; 510/476; 510/473; 510/474; 510/497; 424/70.12; 514/772.3; 514/781; 514/778; 514/774; 514/777; 510/470; 514/772.5; 514/772.4

(57) ABSTRACT

A process for forming a personal care article, the process including (a) producing an extrudate from a twin screw extruder, and (b) forming the extrudate into the personal care article. The personal care article includes (i) from about 10% to about 60% of one or more anionic surfactants, wherein the one or more anionic surfactants have a Krafft point of less than 30° C.; (ii) from about 10% to about 50% of one or more water soluble polymers; (iii) from about 1% to about 30% of one or more plasticizers; and (iv) from about 10% to about 50% water.

### FIELD OF THE INVENTION

The present invention relates to a process of forming a personal care article in the form of an extruded dissolvable solid comprising an anionic surfactant, a water soluble polymer, a plasticizer, and water.

### BACKGROUND OF THE INVENTION

Solid soaps are generally harsh and lead to a squeaky feel on the skin and hair. These qualities are generally unacceptable for many of today's consumers.

Anionic surfactants such as alkyl ether sulfates have been developed to improve upon the disadvantages of solid soaps. However, many anionic surfactants have low Krafft points and are thereby generally formulated only in liquid products. This is one of the primary reasons for the proliferation of liquid shampoos and liquid body washes across the personal care industry. While widely used, liquid products have disadvantages in terms of packaging, storage, transportation, and convenience of use.

To address the disadvantages of liquid products, attempts have been made to incorporate the benefits of low Krafft point anionic surfactants into dissolvable solids. One attempt was to structure the dissolvable solid with one or more water soluble polymers via a casting and drying process. However, this process was energy intensive and costly because it involves the drying of significant amounts of water (typically >50%).

Another attempt was to create porous solids comprising low Krafft point anionic surfactants by freeze-drying. However, freeze-drying was also an energy intensive and costly process.

Producing a dissolvable personal care article via extrusion is a challenge due to the hydrolytic degradation of low Krafft point anionic surfactants under high temperature extrusion conditions. Additionally, low Krafft point anionic surfactants are typically available as aqueous “lamellar” pastes (comprising ˜30% water) and impart significant lubricity inside the extruder barrel which significantly limits the friction and torque between the mixing elements and the extruder barrel, inhibiting the ability of the extruder to work effectively. Moreover, the large viscosity difference between low Krafft point anionic surfactants (as available commercially) and water soluble polymers imposes significant mixing challenges.

Based on the forgoing, there is a need for a dissolvable personal care article comprising low Krafft point anionic surfactants which can be made via a low cost extrusion process.

### SUMMARY OF THE INVENTION

According to an embodiment of the invention, there is provided a process for forming a personal care article comprising (a) producing an extrudate from a twin screw extruder and (b) forming the extrudate into the personal care article, the personal care article comprising (i) from about 10% to about 60% of one or more anionic surfactants, by weight of the personal care article, wherein the one or more anionic surfactants have a Krafft point of less than 30° C.; (ii) from about 10% to about 50% of one or more water soluble polymers, by weight of the personal care article; (iii) from about 1% to about 30% of one or more plasticizers, by weight of the personal care article; and (iv) from about 10% to about 50% water, by weight of the personal care article.

According to another embodiment of the invention, there is provided a process of forming a personal care article comprising (a) adding one or more water soluble polymers and one or more plasticizers to a twin screw extruder to form a premix; (b) heating the premix to from about 150° C. to about 400° C.; (c) cooling the premix to below 135° C.; (d) mixing one or more anionic surfactants water with the premix to form a mixture; (e) extruding the mixture from the twin screw extruder to produce an extrudate; and (f) forming the extrudate into the personal care article, the personal care article comprising (i) from about 10% to about 60% of one or more anionic surfactants, by weight of the personal care wrticle, wherein the one or more anionic surfactants have a Krafft point of less than 30° C.; (ii) from about 10% to about 50% of the water soluble polymer, by weight of the personal care article; (iii) from about 1% to about 30% of the plasticizer, by weight of the personal care article; and (iv) from about 10% to about 50% water, by weight of the personal care article.

These and other features, aspects, and advantages of the invention will become evident to those skilled in the art from a reading of the following disclosure.

### DETAILED DESCRIPTION OF THE INVENTION

While the specification concludes with claims which particularly point out and distinctly claim the invention, it is believed the present invention will be better understood from the following description.

In all embodiments of the present invention, all percentages are by weight of the total composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The term “comprising,” as used herein, means that other steps and other ingredients which do not affect the end result can be added. This term encompasses the terms “consisting of” and “consisting essentially of.” The compositions and methods/processes of the present invention can comprise, consist of, and consist essentially of the elements and limitations of the invention described herein, as well as any of the additional or optional ingredients, components, steps, or limitations described herein.

The term “extruded,” as used herein, means having been produced from the basic components of an extrusion line including a polymer feed, the extruder drive and gear box, the extruder barrel with one or two screws, one or more other injection ports, and the extrusion die. The extruder drive may be electrical in operation and may be geared via a thrust bearing to produce the rotational movement of the one or two extruder screws. The polymer feed to the screw may be from the feed hopper and the feed may be by gravity, metering screw, or simple conveying spiral. The extruder barrel and one or two extruder screws are of high strength steels and are protected from wear and corrosion by a variety of hardening and coating treatments such as nitriding and hard chroming. The extrusion barrel and screw are zoned into between 3 and 15 sections which are individually heated and cooled depending on the material and process parameters. The extrusion die channels the polymer melt from the front of the one or two extruder screws to form the basic shape of the desired product.

The term “Krafft point,” as used herein, (also known as Krafft temperature, or critical micelle temperature) means the minimum temperature at which surfactants may form micelles. Below the Krafft point, there is no value for the critical micelle concentration (CMC), i.e., micelles cannot form. The Krafft point is a point of phase change below which the surfactant remains in crystalline form, even in aqueous solution. The Krafft point is measured experimentally as the temperature (more precisely, narrow temperature range) above which the solubility of a surfactant rises sharply. At this temperature, the solubility of the surfactant becomes equal to the critical micelle concentration. The Krafft point of a surfactant is best determined by locating the abrupt change in slope of a graph of the logarithm of the surfactant' s solubility versus temperature [Source: PAC, 1972, 31, 577 (Manual of Symbols and Terminology for Physicochemical Quantities and Units, Appendix II: Definitions, Terminology and Symbols in Colloid and Surface Chemistry) on page 613].

The term “plasticizer,” as used herein, means any of various substances (typically a solvent) added to a polymer composition to reduce brittleness and to promote plasticity and flexibility.

The term “semi-solid,” as used herein, means a state of matter which is highly viscous and has the qualities of both a solid and a liquid.

The term “solid,” as used herein, means a state of matter wherein the constituents are arranged such that their shape and volume are relatively stable, i.e., not liquid-like or gaseous.

The term “water soluble polymer,” as used herein, includes both water-soluble and water-dispersible polymers, and is defined as a polymer with a solubility in water, measured at 25° C., of at least about 0.1 gram/liter (g/L).

Provided is a process for forming a personal care article comprising (a) producing an extrudate from a twin screw extruder, and (b) forming the extrudate into the personal care article, the personal care article comprising (i) from about 10% to about 60% of one or more anionic surfactants, by weight of the personal care article, wherein the one or more anionic surfactants have a Krafft point of less than 30° C.; (ii) from about 10% to about 50% of one or more water soluble polymers, by weight of the personal care article; (iii) from about 1% to about 30% of one or more plasticizers, by weight of the personal care article; and (iv) from about 10% to about 50% water, by weight of the personal care article.

### Anionic Surfactant

The personal care article may comprise from about 10% to about 60%, alternatively from about 12% to about 50%, and alternatively from about 15% to about 40% of one or more anionic surfactants, by weight of the personal care article. The one or more anionic surfactants may have a Krafft point of less than 30° C., alternatively less than 25° C., alternatively less than 20° C., alternatively less than 15° C., and alternatively less than 10° C.

Non-limiting examples of anionic surfactants may be selected from the group consisting of alkyl sulfates, alkyl ether sulfates, branched alkyl sulfates, branched alkyl alkoxylates, branched alkyl alkoxylate sulfates, alkyloxy alkane sulfonates mid-chain branched alkyl aryl sulfonates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations thereof.

In an embodiment, the one or more anionic surfactants may comprise one or more alkyl ether sulfates according to the following structure:

[chemical structure]

wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
   wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
   wherein M+ is a monovalent counterion selected from a group consisting of sodium, potassium, ammonium, protonated monoethanolamine, protonated diethanolamine, and protonated triethanolamine; and wherein x is on average of from about 0.5 moles to about 3 moles, alternatively from about 1 mole to about 2 moles. In an embodiment, x is on average from about 0.5 moles to about 3 moles of ethylene oxide, alternatively from about 1 mole to about 2 moles of ethylene oxide.

Alkyl sulfates suitable for use herein include materials with the respective formula ROSO₃M, wherein R is an alkyl or an alkenyl of from about 8 carbon atoms to about 24 carbon atoms, and M is a water-soluble cation. Non-limiting examples of M may be selected from the group consisting of ammonium, sodium, potassium, and triethanolamine.

Non-limiting examples of alkyl ether sulfates may be selected from the group consisting of sodium laureth sulfates, ammonium laureth sulfates, potassium laureth sulfates, triethanolamine laureth sulfates, sodium trideceth sulfates, ammonium trideceth sulfates, potassium trideceth sulfates, triethanolamine trideceth sulfates, sodium undeceth sulfates, ammonium undeceth sulfates, potassium undeceth sulfates, triethanolamine undeceth sulfates, and combinations thereof. In an embodiment, the alkyl ether sulfate may be sodium laureth sulfates.

Other suitable anionic surfactants may be described in McCutcheon's Detergents and Emulsifiers, North American Edition (1986), Allured Publishing Corp.; McCutcheon's Functional Materials, North American Edition (1992), Allured Publishing Corp; and U.S. Pat. Nos. 2,486,921, 2,486,922, and 2,396,278.

### Secondary Surfactant

The personal care article may further comprise one or more secondary surfactants selected from the group consisting of amphoteric surfactants, zwitterionic surfactants, and mixtures thereof. The ratio of the one or more anionic surfactants to the one or more secondary surfactants may be from about 15:1 to about 1:2, alternatively from about 10:1 to about 1:1.

Non-limiting examples of amphoteric surfactants may be selected from the group consisting of aliphatic derivatives of secondary and tertiary amines, aliphatic derivatives of heterocyclic secondary and tertiary amines, and mixtures thereof.

Further non-limiting examples of amphoteric surfactants may be selected from the group consisting of sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethy-7 carboxyamphodiacetate, lauraminopropionic acid, lauroamphodipropionic acid, lauryl aminopropylglycine, lauryl diethylenediaminoglycine, and mixtures thereof.

Non-limiting examples of zwitterionic surfactants may be selected from the group consisting of derivatives of secondary and tertiary amines, derivatives of heterocyclic secondary and tertiary amines, derivatives of quaternary ammonium, derivatives of quaternary phosphonium, derivatives of tertiary sulfonium, and mixtures thereof.

Non-limiting examples of zwitterionic surfactants may also be selected from the group consisting of betains including alkyl dimethyl betaine and cocodimethyl amidopropyl betaine, C₈-C₁₈ amine oxides, sulfo and hydroxy betaines, and mixtures thereof.

Further non-limiting examples of zwitterionic surfactants may be selected from the group consisting of cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, oleamidopropyl betaine, coco-sultaine, lauramidopropyl betaine, lauryl betaine, lauryl hydroxysultaine, lauryl sultaine, and mixtures thereof.

### Water-Soluble Polymer

The personal care article may comprise one or more water soluble polymers that may function as a structurant. The personal care article may comprise from about 10% to about 50%, alternatively from about 15% to about 45%, alternatively from about 20% to about 40%, and alternatively from about 25% to about 35% of one or more water soluble polymers, by weight of the personal care article.

The one or more water soluble polymers may have solubility in water, measured at 25° C., of from about 0.1 g/L to about 500 g/L. The one or more water soluble polymers may be of synthetic or natural origin and may be modified by means of a chemical reaction.

In an embodiment, the one or more water soluble polymers may have a weight average molecular weight of from about 40,000 g/mol to about 500,000 g/mol, alternatively from about 50,000 g/mol to about 400,000 g/mol, alternatively from about 60,000 g/mol to about 300,000 g/mol, and alternatively from about 70,000 g/mol to about 200,000 g/mol.

In an embodiment, a 4% by weight solution of one or more water soluble polymers may have a viscosity at 20° C. of from about 4 centipoise to about 80 centipoise, alternatively from about 10 centipoise to about 60 centipoise, and alternatively from about 20 centipoise to about 40 centipoise.

Non-limiting examples of synthetic water soluble polymers may be selected from the group consisting of polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, polyurethanes, polycarboxylic acids, polyvinyl acetates, polyesters, polyamides, polyamines, polyethyleneimines. Further non-limiting examples of synthetic water soluble polymers may be selected from the group consisting of copolymers of anionic, cationic and amphoteric monomers and mixtures thereof, including maleic acrylate based copolymers, maleic methacrylate based copolymers, copolymers of methylvinyl ether and of maleic anhydride, copolymers of vinyl acetate and of crotonic acid, copolymers of vinylpyrrolidone and of vinyl acetate, and copolymers of vinylpyrrolidone and of caprolactam.

Non-limiting examples of natural water soluble polymers may be selected from the group consisting of karaya gum, tragacanth gum, gum arabic, acemannan, konjac mannan, acacia gum, gum ghatti, whey protein isolate, soy protein isolate, guar gum, locust bean gum, quince seed gum, psyllium seed gum, carrageenan, alginates, agar, fruit extracts (pectins), xanthan gum, gellan gum, pullulan, hyaluronic acid, chondroitin sulfate, and dextran, casein, gelatin, keratin, keratin hydrolysates, sulfonic keratins, albumin, collagen, glutelin, glucagons, gluten, zein, shellac, and mixtures thereof.

Non-limiting examples of modified natural water soluble polymers may be selected from the group consisting of (1) cellulose derivatives including hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose, cellulose ethers, cellulose esters; and (2) guar derivatives including hydroxypropyl guar. Suitable hydroxypropylmethylcelluloses may include those available from the Dow Chemical Company (Midland, Mich.).

In an embodiment, the one or more water soluble polymers may be blended with a starch-based material in such an amount as to reduce the overall level of water soluble polymer required. The combined weight percentage of the one or more water soluble polymers and the starch-based material may range from about 10% to about 40%, alternatively from about 12% to about 30%, and alternatively from about 15% to about 25%, by weight of the personal care article. The weight ratio of the one or more water soluble polymers to the starch-based material may range from about 1:10 to about 10:1, alternatively from about 1:8 to about 8:1, alternatively from about 1:7 to about 7:1, and alternatively from about 6:1 to about 1:6.

Non-limiting examples of starch-based materials may be selected from the group consisting of cereals, tubers, roots, legumes, fruits, and combinations thereof. More specifically, non-limiting examples of starch-based materials may be selected from the group consisting of corn, peas, potatoes, bananas, barley, wheat, rice, sago, amaranth, tapioca, arrowroot, canna, sorghum, and combinations thereof. The starch-based materials may also include native starches that are modified using any modification known in the art, including physically modified starches and chemically modified starches.

In order to extrude the one or more water soluble polymers, a plasticizer may be used. Otherwise the one or more water soluble polymers may experience thermal degradation because the melting temperature of the one or more water soluble polymers may be very close to or higher than the one or more water soluble polymers' thermal degradation temperature.

### Plasticizer

The personal care article may comprise one or more plasticizers. The personal care article may comprise from about 1% to about 30%, alternatively from about 5% to about 25%, and alternatively from about 10% to about 20% of one or more plasticizers, by weight of the personal care article. Non-limiting examples of plasticizers may be selected from the group consisting of polyols, copolyols, polycarboxylic acids, polyesters, dimethicone copolyols, and mixtures thereof.

Non-limiting examples of suitable polyols may be selected from the group consisting of glycerin, diglycerin, propylene glycol, ethylene glycol, butylene glycol, pentylene glycol, cyclohexane dimethanol, hexanediol, polyethylene glycol, sorbitol, manitol, lactitol, monohydric and polyhydric low molecular weight alcohols (e.g., C₂-C₈ alcohols), monosaccharides, disaccharides, oligosaccharides, high fructose corn syrup solids, ascorbic acid, and mixtures thereof.

Non-limiting examples of suitable polycarboxylic acids may be selected from the group consisting of citric acid, maleic acid, succinic acid, polyacrylic acid, polymaleic acid, and mixtures thereof.

Non-limiting examples of suitable polyesters may be selected from the group consisting of glycerol triacetate, acetylated-monoglyceride, diethyl phthalate, triethyl citrate, tributyl citrate, acetyl triethyl citrate, acetyl tributyl citrate, and mixtures thereof.

Non-limiting examples of suitable dimethicone copolyols may be selected from the group consisting of PEG-12 dimethicone, PEG/PPG-18/18 dimethicone, and PPG-12 dimethicone.

Further non-limiting examples of suitable plasticizers may be selected from the group consisting of alkyl phthalates, allyl phthalates, napthalates, lactates (e.g., sodium, ammonium and potassium salts), sorbeth-30, urea, lactic acid, sodium pyrrolidone carboxylic acid (PCA), sodium hyaluronate, hyaluronic acid, soluble collagen, modified protein, monosodium L-glutamate, glyceryl polymethacrylate, polymeric plasticizers, proteins, amino acids, hydrogen starch hydrolysates, low molecular weight esters (e.g., esters of C₂-C₁₀ alcohols and acids), and mixtures thereof. In an additional embodiment, non-limiting examples of suitable plasticizers may be alpha and beta hydroxyl acids selected from the group consisting of glycolic acid, lactic acid, citric acid, maleic acid, salicylic acid, and mixtures thereof. EP 0283165 B1 discloses even more suitable plasticizers, including glycerol derivatives such as propoxylated glycerol.

### Water

The personal care article may comprise from about 10% to about 50%, alternatively from about 15% to about 45%, alternatively from about 20% to about 40% water, by weight of the personal care article.

### Benefit Agent

The personal care article may comprise from about 0.1% to about 15% of a benefit agent.

Non-limiting examples of suitable benefit agents may be selected from the group consisting of nonionic surfactants, preservatives, perfumes, coloring agents, cationic polymers, conditioning agents, hair bleaching agents, thickeners, moisturizers, emollients, pharmaceutical actives, vitamins, sunscreens, deodorants, sensates, plant extracts, cosmetic particles, reactive agents, skin lightening agents, skin tanning agents, anti-dandruff agents, exfoliating agents, acids, bases, humectants, enzymes, suspending agents, pH modifiers, hair perming agents, anti-acne agents, anti-microbial agents, exfoliation particles, hair growth agents, insect repellents, chelants, dissolution aids, builders, enzymes, dye transfer inhibiting agents, softening agents, and mixtures thereof.

In an embodiment, the personal care article may be configured as a lubricating strip on a disposable shaving device.

### Conditioning Agents

Non-limiting examples of conditioning agents may be selected from the group consisting of silicones, organic oils,and mixtures thereof. Non-limiting examples of silicones may be selected from the group consisting of silicone oils, high molecular weight polyalkyl or polyaryl siloxanes, aminosilicones, cationic silicones, silicone gums, high refractive silicones, low molecular weight polydimethyl siloxanes, silicone resins, and mixtures thereof. Non-limiting examples of organic oils may be selected from the group consisting of hydrocarbon oils, polyolefins, fatty esters, and mixtures thereof. Additional non-limiting examples of conditioning agents and optional suspending agents for silicone may be found in U.S. Pat. Nos. 5,104,646 and 5,106,609, which are incorporated herein by reference.

The silicone gums and the high molecular weight polyalkyl or polyaryl siloxanes may have a viscosity of from about 100,000 mPa·s to about 30,000,000 mPa·s, alternatively from about 200,000 mPa·s to about 30,000,000 mPa·s. The silicone gums and the high molecular weight polyalkyl or polyaryl siloxanes may have a molecular weight of from about 100,000 g/mol to about 1,000,000 g/mol, and alternatively from about 120,000 g/mol to about 1,000,000 g/mol.

The low molecular weight polydimethyl siloxanes may have a viscosity of from about 1 mPa·s to about 10,000 mPa·s at 25° C., and alternatively from about 5 mPa·s to about 5,000 mPa·s. The low molecular weight polydimethyl siloxanes may have a molecular weight of from about 400 to about 65,000, and alternatively from about 800 to about 50,000.

In an embodiment, the conditioning agent may include one or more aminosilicones. Aminosilicones may be silicones containing at least one primary amine, secondary amine, tertiary amine, or a quaternary ammonium group. In an embodiment the aminosilicones may have less than 0.5% nitrogen by weight of the aminosilicone, in another embodiment less than 0.2%, in yet another embodiment less than 0.1%.

In an embodiment, the aminosilicones may have a particle size of less than 50m once incorporated into the final composition. The particle size measurement may be taken from dispersed droplets in the final composition. Particle size may be measured by means of a laser light scattering technique using a Horiba model LA-910 Laser Scattering Particle Size Distribution Analyzer (Horiba Instruments, Inc.).

The aminosilicones may have a viscosity of from about 1,000 cs (centistokes) to about 1,000,000 cs, in another embodiment from about 10,000 cs to about 700,000 cs, in yet another embodiment from about 50,000 cs to about 500,000 cs, and in yet another embodiment from about 100,000 cs to about 400,000 cs. This embodiment may also comprise a low viscosity fluid. The viscosity of aminosilicones discussed herein is measured at 25° C.

In another embodiment, the aminosilicones may have a viscosity of from about 1,000 cs to about 100,000 cs, in another embodiment from about 2,000 cs to about 50,000 cs, in another embodiment from about 4,000 cs to about 40,000 cs, and in yet another embodiment from about 6,000 cs to about 30,000 cs.

The personal care composition may comprise from about 0.05% to about 20%, alternatively from about 0.1% to about 10%, and alternatively from about 0.3% to about 5% aminosilicones by weight of the personal care composition.

### Anti-Dandruff Agents

In an embodiment, the personal care article may comprise an anti-dandruff agent which may be an anti-dandruff particulate. Non-limiting examples of suitable anti-dandruff agents may be selected from the group consisting of pyridinethione salts, azoles (e.g. ketoconazole, econazole, and elubiol), selenium sulphide, particulate sulfur, keratolytic agents (e.g. salicylic acid), and mixtures thereof. In an embodiment, the anti-dandruff agent is a pyridinethione salt.

Pyridinethione salt particulates are suitable particulate anti-dandruff agents. In an embodiment, the anti-dandruff agent may be a 1-hydroxy-2-pyridinethione salt in particulate form. The personal care article may comprise from about 0.01% to about 5%, alternatively from about 0.1% to about 3%, and alternatively from about 0.1% to about 2% pyridinethione salt particulates. In an embodiment, the pyridinethione salt particulates may be those formed from heavy metals such as zinc, tin, cadmium, magnesium, aluminium, and zirconium. In any embodiment, the pyridinethione salt may be the zinc salt of 1-hydroxy-2-pyridinethione (known as “zinc pyridinethione” or “ZPT”) optionally in platelet particle form. In an embodiment, the zinc salt of 1-hydroxy-2-pyridinethione in platelet particle form may have an average particle size of less than 20 microns, alternatively less than 5 microns, and alternatively less than 2.5 microns. Salts formed from other cations, such as sodium, may also be suitable anti-dandruff agents. Pyridinethione anti-dandruff agents are described, for example, in U.S. Pat. Nos. 4,323,683; 4,379,753; and 4,470,982.

The personal care article may also comprise an antimicrobial active. Non-limiting examples of suitable anti-microbial actives may be selected from the group consisting of coal tar, sulfur, charcoal, aluminum chloride, gentian violet, octopirox (piroctone olamine), ciclopirox olamine, undecylenic acid and its metal salts, potassium permanganate, selenium sulphide, sodium thiosulfate, propylene glycol, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, azoles, and mixtures thereof. Further non-limiting examples of suitable anti-microbial agents may be selected from the group consisting of itraconazole, ketoconazole, selenium sulphide, coal tar, and mixtures thereof.

In an embodiment, the anti-microbial agent may be an imidazole selected from the group consisting of benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof. In an embodiment, the anti-microbial agent may be a triazole selected from the group consisting of terconazole, itraconazole, and mixtures thereof.

### Cationic Polymers

In an embodiment, the personal care article may comprise a cationic polymer. Cationic polymers useful herein may include those discussed in US 2007/0207109 A1 and US 2008/0206185 A1, such as synthetic copolymers of sufficiently high molecular weight to effectively enhance the deposition of the conditioning active components of the personal care article described herein. Combinations of cationic polymer may also be utilized. The average molecular weight of the synthetic copolymers is generally between about 10,000 and about 10 million, preferably between about 100,000 and about 3 million, still more preferably between about 200,000 and about 2 million.

In a further embodiment, the synthetic copolymers have mass charge densities of from about 0.1 meq/gm to about 6.0 meq/gm, alternatively from about 0.5 meq/gm to about 3.0 meq/gm, at the pH of intended use of the personal care article. The pH may be from about pH 3 to about pH 9, and alternatively from about pH 4 and about pH 8.

In yet another embodiment, the synthetic copolymers have linear charge densities from at least about 2 meq/A to about 500 meq/A, and more preferably from about 20 meq/A to about 200 meq/A, and most preferably from about 25 meq/A to about 100 meq/A.

Cationic polymer may be copolymers or homopolymers. In one embodiment, a homopolymer is utilized in the present composition. In another embodiment, a copolymer is utilized in the present composition. In another embodiment a mixture of a homopolymer and a copolymer is utilized in the present composition. In another embodiment, a homopolymer of a naturally derived nature, such as cellulose or guar polymer discussed herein, is combined with a homopolymer or copolymer of synthetic origin, such as those discussed below.

Homopolymers—Non-crosslinked cationic homopolymers of the following monomers are also useful herein: 3-acrylamidopropyltrimethylammonium chloride (APTAC), diallyldimethylammonium chloride (DADMAC), [(3- methylacrylolyamino)propyl]trimethylammonium chloride (MAPTAC), 3-methyl-1-vinylimidazolium chloride (QVI); [2-(acryloyloxy)ethyl]trimethylammonium chloride and [2-(acryloyloxy)propyl]trimethylammonium chloride.

Copolymers—copolymer may be comprises of two cationic monomer or a nonionic and cationic monomers.

The personal care articles may also comprise cellulose or guar cationic deposition polymers. Generally, such cellulose or guar cationic deposition polymers may be present at a concentration from about 0.05% to about 5%, by weight of the composition. Suitable cellulose or guar cationic deposition polymers have a molecular weight of greater than about 5,000. Additionally, such cellulose or guar deposition polymers have a charge density from about 0.5 meq/g to about 4.0 meq/g at the pH of intended use of the personal care article, which pH will generally range from about pH 3 to about pH 9, preferably between about pH 4 and about pH 8. The pH of the compositions is measured neat.

In one embodiment of the invention, the cationic polymers are derivatives of Hydroxypropyl Guar, examples of which include polymers known via the INCI nomenclature as Guar Hydroxypropyltrimonium Chloride, such as the products sold under the name Catinal CG-100, Catinal CG-200 by the company Toho, Cosmedia Guar C-261N, Cosmedia Guar C-261N, Cosmedia Guar C-261N by the company Cognis, DiaGum P 5070 by the company Freedom Chemical Diamalt, N-Hance Cationic Guar by the company Hercules/Aqualon, Hi-Care 1000, Jaguar C-17, Jaguar C-2000, Jaguar C-13S, Jaguar C-14S, Jaguar Excel by the company Rhodia, Kiprogum CW, Kiprogum NGK by the company Nippon Starch.

### Process of Forming the Personal Care Article

The process of forming a personal care article may comprise (a) adding one or more water soluble polymers and one or more plasticizers to a twin screw extruder to form a premix; (b) heating the premix to from about 150° C. to about 400° C.; (c) cooling the premix to below 135° C.; (d) mixing one or more anionic surfactants water with the premix to form a mixture; (e) extruding the mixture from the twin screw extruder to produce an extrudate; and (f) forming the extrudate into the personal care article.

The process of forming a personal care article may comprise adding one or more water soluble polymers and one or more plasticizers to a twin screw to form a premix, and heating the premix to from about 150° C. to about 400° C., alternatively from about 155° C. to about 300° C., and alternatively from about 160° C. to about 250° C. In an embodiment, the one or more water soluble polymers and the one or more plasticizers may be compounded together by a separate extrusion process and then added to the twin screw extrusion process as a single ingredient. In another embodiment, the one or more water soluble polymers and the one or more plasticizers may be added to the twin screw extrusion process as separate ingredients. In an embodiment, a twin-screw extruder from Leistritz (with 27 mm screw diameter, 40:1 L/D ratio, 10 independent temperature control barrel pieces) may be used.

The process of forming a personal care article may comprise cooling the premix to below 135° C., alternatively below about 130° C., alternatively below about 125° C., and alternatively below about 120° C., and then mixing one or more anionic surfactants with the premix to form a mixture. The water may enter the process as a component of one or more raw materials comprising the anionic surfactants, by separate addition to the process, or a combination thereof.

The process of forming a personal care article may comprise forming the extrudate into the personal care article as described above. In an embodiment, personal care articles of differing shapes or designs are obtained via thermoforming. In this operation, the extrudate exits the extruder at a hot enough temperature, or is later heated to such a temperature, at which it is pliable or shapeable and then forced against a mold by applying mechanical pressure, a vacuum, or air. The extrudate is later cooled and removed from the mold. The extrudate may be malleable upon leaving the extruder and well suited for the thermoforming step.

Other methods of forming the extrudate into the personal care article may be used in addition to the thermoforming operations discussed above. Non-limiting examples of such methods may be selected from the group consisting of injection molding, blow molding, extrusion-blow molding, stamping, and combinations thereof.

The extrudate may also be shaped via die geometry, cutting or molding processes. In one embodiment, the die may be cylindrical die, an annular die, or a slot die. In one embodiment, the extrudate may be cut into defined lengths including tubules, bars, films, pellets, spheres, hollow tubules, hollow bars, etc. In another embodiment, two or more different extrudates may be combined into a shape via co-extruding, co-mixing or layering. In an embodiment, the combined extrudates may comprise differing compositions.

In an embodiment, a twin screw extrusion process, either alone or in combination with other forming operations, may be used depending on the desired type of the final product. Two different types of extruders may be employed consisting of a twin screw extruder and single screw extruder. The twin screw extruder may be a conical twin screw extruder. In an embodiment, the process may utilize a tandem extrusion set up which consists of two or more of extruders connected in a series or in parallel. The tandem extrusion set up may use a twin-screw extruder to improve mixing between the water soluble polymer and the rest of ingredients, followed by a single-screw extruder for effective cooling.

The resulting extrudate leaving the extruder may be in a rope or cylindrical form. By varying the size and configuration of the die opening of the extruder, different forms such as sheets or slabs of varying thickness and widths, irregular profiles, and other cross sectional shapes can be obtained. These can be cut into a multiplicity of regular individual solids via a number of means either as the extrudate exits the extruder or via a subsequent post-processing step.

In an embodiment, a further third zone temperature may be employed involving further cooling of the mixture prior to exiting the extruder or via a secondary tandem extruder. The third zone temperature range may be from about 50° C. to about 110° C., alternatively from about 60° C. to about 100° C., and alternatively from about 70° C. to about 90° C.

### EXAMPLES

### 1^{st} Extrusion Compounding of Polyvinyl Alcohol (PVOH)/Glycerin

Polyvinyl alcohol may be a water soluble polymer and may provide structural integrity for the personal care article. In order to extrude PVOH, a plasticizer may be used; otherwise the PVOH may experience thermal degradation because the melting temperature of PVOH may be very close to or higher than its thermal degradation temperature. For this purpose, liquid-form glycerin may be utilized as a plasticizer. However, other plasticizers may also be employed to help suppress the processing temperature of PVOH below its thermal degradation temperature. Glycerin may be injected into the extruder barrel as early as possible in this compounding process.

Since the ration of glycerin to PVOH may determine the possible processing temperature range and manufacturability of the premix, the appropriate weight ratio between glycerin and PVOH may be determined based on the results from a Differential Scanning calorimeter (DSC). Although the melting temperature of PVOH may be further decreased with increased glycerin content, the post processing-ability of high glycerin premixes may not be suitable for a two-stage manufacturing strategy. Therefore, the specific weight ratio between PVOH and glycerin in this embodiment is 66.7 wt % to 33.3 wt %, respectively.

Once the weight ratio of PVOH and glycerin is determined, the extrusion compounding may be carried out. The use of a twin-screw extruder may not only provide a significant amount of mechanical mixing energy via shear and extension of materials but may also provide flexibility to optimize the configuration of extrusion screw based on the requirements of different formulations. For this purpose, both co-rotation and counter-rotation types of twin-screw extruders may be implemented. Further, a co-rotation or a counter-rotation twin-screw extruder may be equipped with multiple injection ports along its barrel in order to directly inject liquid-form glycerin into the barrel of the extruder.

In an embodiment, a twin-screw extruder from Leistritz (with 27mm screw diameter, 40:1 L/D ratio, 10 independent temperature control barrel pieces) may be used. The twin-screw extrusion system may be heated to desired temperatures prior to the actual compounding process. Sample temperatures are shown in Table 1 below.

TABLE 1
Temperature Profile ofTwin-Screw Extruder
(TSE) for Compounding of PVOH and Glycerin

| Barrel zone | Temperature [° C.] |
|---|---|
| Zone 1 | 220 |
| Zone 2 | 240 |
| Zone 3 | 200 |
| Zone 4 | 200 |
| Zone 5 | 180 |
| Zone 6 | 180 |
| Zone 7 | 160 |
| Zone 8 | 160 |
| Zone 9 | 150 |
| Die | 140 |

When the temperatures are reached, waiting about ten minutes before the operation may ensure the heat is transferred from the barrel to the screw. This time may be varied depend on the types, sizes, and makers of extruders. The PVOH granulates may be fed into the barrel using a weight-loss gravimetric feeder from Brabender Technologies whereas the glycerin may be injected into the barrel using 260D high pressure syringe pumps from Teledyne Isco, which may be adjusted based on the feeding rate of PVOH. Although other types of high pressure liquid pumps may be used to inject liquid-form glycerin, the 260D high pressure syringe pump may provide an injection pressure of up to 6500 psi and accurate volumetric flow rates. It is also possible to use different processing temperatures with the given weight ratio of PVOH and glycerin. However, excessively high torque may be required if the processing temperatures are lower than the values in Table 1. Also, if the processing temperatures are higher than the values in Table 1, thermal degradation of PVOH may occur during the extrusion compounding process.

Because both PVOH and glycerin are water-soluble materials, the composite may be cooled with compressed air, which may have a temperature range of from about 10° C. to about 20° C., as it extrudes out from the extruder. There may be other means to cool the water-soluble composite, but compressed air may be a cost effective method. The final stage of compounding may be pelletizing of the cooled PVOH and glycerin premix, and this step may be carried out using a pellletizer, which may be BT25 from Scheer Bay Co. or any type of commercially available pelletizer.

### 2^{nd} Extrusion Compounding of PVOH/Glycerin and an Anionic Surfactant Solution Mixture

The second extrusion compounding process may effectively mix a PVOH/glycerin matrix with an anionic surfactant solution mixture. Similar to the first extrusion compounding process, the second extrusion compounding may be carried out with a single-screw extruder, a twin-screw extruder, or a tandem extrusion line. A tandem extrusion line, which consists of a twin-screw extruder and a single-screw extruder, may provide not only a sufficient mixing from the twin-screw extruder but also effective cooling from the single-screw extruder. Furthermore, the twin-screw extruder may have a number of injection ports where the additional water and the surfactant solution mix may be injected into the barrel, and the injection locations may affect the mixing quality of the final extrudate.

In an embodiment, the twin-screw extruder with 27mm screw diameter and 40:1 L/D ratio from Leistritz may be utilized. The single-screw extruder with 0.75″ of screw diameter and 30 L/D ratio may also be employed. These two extruders may be connected using a flange pipe, which may have an independent temperature control unit. The set temperature values for the tandem extrusion setup are described in Table 2 below. In one embodiment, the processing temperature may be below about 120° C. after injecting the surfactant solution. If the set temperatures are higher, the thermal degradation of 70% active alkyl ether sulfate may be triggered. If the temperatures are lower, it may not melt the PVOH/glycerin matrix, which may result in poor mixing behavior.

TABLE 2
Temperature Profile of Tandem Extrusion
Line for Compounding of Solid Bar

| Barrel zone | Temperature [° C.] |
|---|---|
| TSE Zone 1 | 140 |
| TSE Zone 2 | 160 |
| TSE Zone 3 | 120 |
| TSE Zone 4 | 120 |
| TSE Zone 5 | 120 |
| TSE Zone 6 | 110 |
| TSE Zone 7 | 100 |
| TSE Zone 8 | 100 |
| TSE Zone 9 | 90 |
| TSE Adaptor | 90 |
| Flange Pipe | 100 |
| SSE Barrel 1 | 90 |
| SSE Barrel 2 | 85 |
| SSE Barrel 3 | 80 |
| SSE Adaptor | 75 |

For the second extrusion compounding process, there may be two injection locations, one for the surfactant solution mixture and another for the additional water. The surfactant solution mixture may be injected into either Zone 3 or Zone 6. Any additional water may be injected into Zone 6 if the surfactant solution mixture is injected into Zone 3 or vice versa. The injection locations being away from each other may provide enough residence time for the first injected material to react with the PVOH/glycerin matrix before getting mixed with the second injected material. In the case of the surfactant solution mixture, it may be possible to inject each ingredient at different locations separately rather than as the surfactant solution mixture at one specific location. However, injecting as the surfactant solution mixture may provide more convenience to the manufacturing process. Both the additional water and the surfactant solution mixture may be injected into the twin-screw extruder barrel using three or more high pressure syringe pumps, 260D pumps from Teledyne Isco Inc. In addition, the PVOH/glycerin pellets may be fed into the barrel using a weight-loss gravimetric feeder from Brabender Technologies.

At the end of the second extrusion compounding process, two methods may be used to form the extrudate into a bar shaped personal care article. First, three-piece plastic molds may be used. The three-piece plastic mold may have bottom, top, and cover pieces. During the extrusion process, the top and bottom pieces may be assembled together and may be filled with the extrudate. In order to use this three-piece mold, a custom-made adaptor may be utilized at the end of the single screw extruder or in front of the die. Once the mold is filled, the cover piece may be put on and the entire mold may be wrapped with a commercial thin-film transparent plastic wrap. Other methods may be implemented to prevent the water loss during the cooling stage, but the utilization of commercially available thin plastic film may be cost effective. The extrudate within the mold may be cooled overnight at room temperature and then the sample may be taken out from the mold for evaluation. In order to achieve desired quality of personal care article, other cooling methods may be employed. In an embodiment, cooling in the temperature lower than 0° C. may be proposed to increase the hardness of personal care article as well as to reduce the required cooling time.

Second, a sheet extrusion die may be implemented to form the extrudate into bar shaped personal care articles. The sheet extrusion may provide more flexibility and versatility to the possible designs of the final product. Since this process may integrate the cooling phase as the extrudate comes out from the die, it may be possible to reduce the manufacturing time as well. Furthermore, the implementation of this continuous manufacturing technology may be more cost effective than the manufacturing with three-piece mold. The design may be based on the die back pressure and the extrusion throughput. Additional molding methods are also possible including extrusion depositing into molds, injection molding, or post-heating molding of the extrudate.

### Use of Additional Water

For the second extrusion compounding process, the existence of additional water and its content may play a critical role in determining the quality of the extrudate. Additional water may serve as another plasticizer for the PVOH/glycerin matrix as well as a mixing enhancer of the extrudate. Although some ingredients such as alkyl ether sulfate and cocamidopropyl betaine may have a significant amount of water in their solution states already, the water's participation in the plasticization and mixing may be very minimal. Therefore, use of additional water may be required to produce the personal care article with desired properties.

Additional water may also affect the hardness of the extrudate. If the additional water content is excessively high, then the final composite may not have enough structural integrity to maintain the final shape. On the other hand, excessively low content of additional water may deteriorate mixing quality.

### PRODUCT EXAMPLES

### Example 1

### Extrusion without Additional Water

For this example, PVOH/glycerin pellets are prepared via the first extrusion compounding process as described above. For the second extrusion compounding process, however, the additional water is not utilized to observe its influence on the final dissolving solid composite.

As for the surfactant solution mixture, it consists of alkyl ether sulfates and perfume only. The materials contents are provided in Table 3. When calculating overall water content for all the examples described in this invention, it is assumed that alkyl ether sulfates include 30% water and perfume does not have any water.

TABLE 3
Material Ingredients

| Component | wt % |
|---|---|
| Polyvinyl alcohol | 35.09 |
| Glycerin | 17.54 |
| Alcohol ethoxy sulfate solution (70% activity) | 44.91 |
| Additional Water | 0.00 |
| Perfume | 2.46 |
| Overall Water | 13.47 |
| Polymer:Active Surfactant | 52.7:47.3 |
| Active alcohol ethoxy sulfate | 31.44 |

For this example, the tandem extrusion set up is employed as described previously. In addition, a sheet extrusion die is implemented for shaping the extrudate into a personal care article. The temperature profile of tandem extrusion for this example is shown in Table 4 below. The single-screw barrel temperatures may be slightly lower than those in Table 2 in order to build up sufficient pressure before the die. The solution mix is injected in TSE Zone 3 using a set of 260D high pressure syringe pumps from Teledyne Isco. In this example, a lack of additional water results in poor mixing between the PVOH/glycerin matrix and the anionic surfactant solution.

TABLE 4
Temperature profile of tandem extrusion
line for compounding of solid bar

| Barrel zone | Temperature [° C.] |
|---|---|
| TSE Zone 1 | 140 |
| TSE Zone 2 | 160 |
| TSE Zone 3 | 120 |
| TSE Zone 4 | 120 |
| TSE Zone 5 | 120 |
| TSE Zone 6 | 110 |
| TSE Zone 7 | 110 |
| TSE Zone 8 | 100 |
| TSE Zone 9 | 90 |
| TSE Adaptor | 90 |
| Flange Pipe | 90 |
| SSE Barrel 1 | 75 |
| SSE Barrel 2 | 65 |
| SSE Barrel 3 | 65 |
| SSE Adaptor | 60 |
| Sheet Die | 30 |

### Example 2

### Extrusion with the Minimum Additional Water

In this example, 20 phr (parts per hundred parts of polymer resin material added to the feeder) of additional water is utilized for the second extrusion compounding process. The implementation of additional water may enable the final product to maintain its structural integrity in a long bar shape. The additional water is injected into Zone 6 of the twin-screw extruder. The surfactant solution mixture is injected into Zone 3 as aforementioned in Example 1. The rest of processing variables including the temperature profile of the tandem extrusion setup and the sheet extrusion die are identical to Example 1. The material contents are shown in Table 5 below.

TABLE 5
Material Ingredients

| Component | wt % |
|---|---|
| Polyvinyl alcohol | 32.79 |
| Glycerin | 16.39 |
| Alcohol ethoxy sulfate solution | 41.97 |
| (70% activity) | |
| Additional Water | 6.56 |
| Perfume | 2.30 |
| Overall Water | 19.15 |
| Polymer:Active Surfactant | 52.7:47.3 |
| Active alcohol ethoxy sulfate | 29.38 |

### Example 3

### Extrusion with Medium Level of Additional Water

For this example, the content of additional water is increased by two times from the minimum amount of Example 2. The rest of manufacturing conditions are identical to those of Example 2. Although the additional water content is increased by two folds, the quality of final product may be very similar to that of Example 2. Due to the increased water content, the final product becomes slightly more transparent than the final product of Example 2. The contents of materials are illustrated in Table 6.

TABLE 6
Material Ingredients

| Component | Wt % |
|---|---|
| Polyvinyl alcohol | 30.77 |
| Glycerin | 15.38 |
| Alcohol ethoxy sulfate solution | 39.38 |
| (70% activity) | |
| Additional Water | 12.31 |
| Perfume | 2.15 |
| Overall Water | 24.12 |
| Polymer:Active Surfactant | 52.7:47.3 |
| Active alcohol ethoxy sulfate | 27.57 |

### Example 4

### Extrusion with Appropriate Hardness

In this example, 30% active cocamidopropyl betaine (CAPB) is introduced to the surfactant solution mixture. Prior to the second extrusion compounding of this example, therefore, cocamidopropyl betaine is added and mixed with alkyl ether sulfate and perfume. The contents of materials are shown in Table 7. It is assumed that cocamidopropyl betaine includes of 70 wt % of water and this assumption applies to the rest of examples. In this example, the surfactant solution consists of alkyl ether sulfate, cocamidopropyl betaine, and perfume.

TABLE 7
Material Ingredients

| Component | wt % |
|---|---|
| Polyvinyl alcohol | 28.57 |
| Glycerin | 14.29 |
| Alcohol ethoxy sulfate solution | 27.97 |
| (70% activity) | |
| Cocamidopropyl betaine solution | 4.94 |
| (30% activity) | |
| Additional Water | 22.86 |
| Perfume | 1.37 |
| Overall Water | 34.71 |
| Polymer:Surfactant Solution | 57.6:42.4 |
| Active alcohol ethoxy sulfate | 19.58 |
| Active cocamidopropyl betaine | 1.48 |
| Active SLE1S:Active CAPB | 93:7 |

For the second extrusion compounding of this example, the surfactant solution mixture is injected at Zone 3 whereas the additional water is injected at Zone 6. This example may implement a three-piece mold method for the shaping of the personal care article. The temperature profile is provided in Table 2. The final products of this example may have the appropriate hardness and texture. Furthermore, the samples may not have stickiness on the surfaces.

### Example 5

### Extrusion with Excessively Low Hardness

This example incorporates increased additional water content and increased amount of surfactant solution mixture from those of Example 4. According to the contents of materials shown in Table 8, the additional water content is increased by about 7.05 wt % while the surfactant solution mix content is increased by 7.78 wt % from Example 4. As a result, the content of polyvinyl alcohol is decreased by 9.88 wt %.

As described in Example 4, the surfactant solution mixture is injected at Zone 3 and the additional water is injected at Zone 6 of the twin-screw extruder. The temperature profile of this example is exhibited in Table 2 and the three-piece mold method is employed. The final products may not able to maintain their shapes during the cooling and may shrink.

TABLE 8
Material Ingredients

| Component | wt % |
|---|---|
| Polyvinyl alcohol | 18.69 |
| Glycerin | 9.35 |
| Alcohol ethoxy sulfate solution | |
| (70% activity) | 34.32 |
| Cocamidopropyl betaine solution | 6.06 |
| (30% activity) | |
| Additional Water | 29.91 |
| Perfume | 1.68 |
| Overall Water | 44.45 |
| Polymer:Surfactant Solution | 42.0:58.0 |
| Active alcohol ethoxy sulfate | 24.02 |
| Active cocamidopropyl betaine | 1.82 |
| Active SLE1S:Active CAPB | 93:7 |

### Example 6

### Extrusion with High Hardness

This example presents the case when both the additional water content and the surfactant solution mix content is decreased from Example 4. They decrease by 3.51 wt % and 2.03 wt %, respectively. Table 9 below shows the contents of materials for this example, and the identical processing conditions as Example 4 and 5 are applied. The final products may maintain their final shape during the cooling phase. In terms of hardness, the final products of Example 6 may be considerably harder than those of Example 4.

TABLE 9
Material Ingredients

| Component | Wt % |
|---|---|
| Polyvinyl alcohol | 32.26 |
| Glycerin | 16.13 |
| Alcohol ethoxy sulfate solution | 26.32 |
| (70% activity) | |
| Cocamidopropyl betaine solution | 4.64 |
| (30% activity) | |
| Additional Water | 19.35 |
| Perfume | 1.29 |
| Overall Water | 30.49 |
| Polymer:Surfactant Solution | 61.9:38.1 |
| Active alcohol ethoxy sulfate | 18.42 |
| Active cocamidopropyl betaine | 1.39 |
| Active SLE1S:Active CAPB | 93:7 |

### Example 7

### Extrusion with Increased CAPB with respect to SLE1S

This example utilizes a larger amount of cocamidopropyl betaine with respect to the amount of SLE1S when compared to Example 4 through 6. For those three examples, the weight ratio of SLE1S to CAPB is 85 to 15, respectively. In this example, the weight ratio between SLE1S and CAPB is 75 to 25, respectively. The material contents are shown in Table 10 below and the identical processing conditions as Example 4 through 6 are implemented. The final products may hold their shapes adequately and the stickiness on the surface may not be noticeable. Furthermore, the hardness of the final product may be acceptable, which is very close to that of Example 4.

TABLE 10
Material Ingredients

| Component | wt % |
|---|---|
| Polyvinyl alcohol | 28.57 |
| Glycerin | 14.29 |
| Alcohol ethoxy sulfate solution | 24.21 |
| (70% activity) | |
| Cocamidopropyl betaine solution | 8.07 |
| (30% activity) | |
| Additional Water | 22.86 |
| Perfume | 2.00 |
| Overall Water | 35.77 |
| Polymer:Surfactant Solution | 59.6:40.4 |
| Active alcohol ethoxy sulfate | 16.95 |
| Active cocamidopropyl betaine | 2.42 |
| Active SLE1S:Active CAPB | 87.5:12.5 |

### Example 8

### Extrusion with Further Increased CAPB with respect to SLE1S

For this example, the content of CAPB is further increased with respect to SLE1S when compared to that of Example 7. In Example 7, the weight ratio between SLE1S and CAPB is 75 to 25, respectively. In this example, however, the weight ratio of SLE1S and CAPB is 67 to 33, respectively. The contents of materials are exhibited in Table 11 below and the identical processing conditions as Example 4 through 7 are employed for this example as well. The stickiness on the surface of the final product may be increased. In addition, the samples may experience shrinkage during the cooling stage.

TABLE 11
Material Ingredients

| Component | wt % |
|---|---|
| Polyvinyl alcohol | 25.64 |
| Glycerin | 12.82 |
| Alcohol ethoxy sulfate solution | 19.41 |
| (70% activity) | |
| Cocamidopropyl betaine solution | 9.56 |
| (30% activity) | |
| Additional Water | 30.77 |
| Perfume | 1.79 |
| Overall Water | 43.29 |
| Polymer:Surfactant Solution | 60.9:39.1 |
| Active alcohol ethoxy sulfate | 13.59 |
| Active cocamidopropyl betaine | 2.87 |
| Active SLE1S:Active CAPB | 82.6:17.4 |

### Example 9

### Extrusion with Amino Silicone Fluid and Injection Location Change

In this example, amino silicone fluid is introduced for the first time as a part of the final composite. Thus, the surfactant solution mixture of this example consists of alkyl ether sulfate, cocamidopropyl betaine, perfume, and amino silicone fluid. The weight ratio of SLE1S and CAPB is 75 to 25, respectively, as described in Example 7. The contents of materials are shown in Table 12 below.

TABLE 12
Material Ingredients

| Component | wt % |
|---|---|
| Polyvinyl alcohol | 27.44 |
| Glycerin | 13.72 |
| Alcohol ethoxy sulfate solution | 23.25 |
| (70% activity) | |
| Cocamidopropyl betaine solution | 7.75 |
| (30% activity) | |
| Additional Water | 21.95 |
| Perfume | 1.92 |
| Amino Silicone Fluid | 3.96 |
| Overall Water | 34.35 |
| Polymer:Active Surfactant | 59.6:40.4 |
| Active alcohol ethoxy sulfate | 16.28 |
| Active cocamidopropyl betaine | 2.33 |
| Active SLE1S:Active CAPB | 87.5:12.5 |

For Example 2 through 8, the surfactant solution mix is injected at Zone 3, whereas the additional water is injected at Zone 6 of the twin-screw extruder. In the case of this example, however, the injection locations are switched. In other words, the additional water is injected at Zone 3 meanwhile the surfactant solution is injected at Zone 6. The rest of processing conditions stay identical to those of Example 4 through 8.

The final samples of this example may exhibit stickiness on the surfaces and may adhere to the molds during the cooling stage. This stickiness may increase the difficulty when taking out the samples from the molds.

### Example 10

### Extrusion with Amino Silicone and Increased Cocamidopropyl Betaine

In this example, similar to Example 8, the weight ratio of cocamidopropyl betaine is increased with respect to the weight of alkyl ether sulfate. The weight ratio between these two in Example 9 is 75 to 25, but it becomes 67 to 33 in this example. Furthermore, amino silicone fluid is employed again as Example 9. The contents of materials are exhibited in Table 13 below and the rest of processing conditions are identical to those of Example 9 including the injection locations of the additional water and the surfactant solution mix.

TABLE 13
Material Ingredients

| Component | wt % |
|---|---|
| Polyvinyl alcohol | 27.44 |
| Glycerin | 13.72 |
| Alcohol ethoxy sulfate solution | 20.77 |
| (70% activity) | |
| Cocamidopropyl betaine solution | 10.23 |
| (30% activity) | |
| Additional Water | 21.95 |
| Perfume | 1.92 |
| Amino Silicone Fluid | 3.96 |
| Overall Water | 35.34 |
| Polymer:Active Surfactant | 60.9:39.1 |
| Active alcohol ethoxy sulfate | 14.54 |
| Active cocamidopropyl betaine | 3.07 |
| Active SLE1S:Active CAPB | 82.6:17.4 |

Due to this weight ratio change, the content of cocamidopropyl betaine increases to 10.23 wt % from 7.75 wt % of Example 9 while the content of alkyl ether sulfate decreases to 20.77 wt % from 23.25 wt % of Example 9.

The final dissolving solid bars of this example may be able to maintain their shape adequately. When the samples are taken out from the molds, it may be more difficult to take them out when compared to the cases of Example 9.

### Example 11

### Extrusion with Higher Temperature than Thermal Degradation Temperature of Alkyl Ether Sulfate

This example is a comparative example falling outside of the claimed invention. An alkyl ether sulfate is injected into the twin-screw extruder and is processed at a higher temperature range. This example employs alkyl ether sulfate for the surfactant solution mix. During the second extrusion compounding process, the additional water is not be injected. The contents of materials are provided in Table 14 below.

TABLE 14
Material Ingredients

| Component | wt % |
|---|---|
| Polyvinyl alcohol | 23.53 |
| Glycerin | 11.77 |
| Alcohol ethoxy sulfate solution | 64.7 |
| (70% activity) | |
| Overall Water | 19.41 |
| Polymer:Active Surfactant | 34.2:65.8 |
| Active alcohol ethoxy sulfate | 45.29 |

For this example, the second extrusion compounding process is carried out using a twin-screw extruder instead of a tandem extrusion line in order to minimize the time that alkyl ether sulfate is exposed to high temperature environment. Alkyl ether sulfate is injected at Zone 6 of the twin-screw extruder and the temperature profile of the second extrusion compounding process is shown in Table 15 below. The set temperature values are higher than the thermal degradation temperature of alkyl ether sulfate, which is close to about 120° C.

TABLE 15
Temperature Profile of Second
Compounding in Example 11

| Barrel zone | Temperature [° C.] |
|---|---|
| Zone 1 | 130 |
| Zone 2 | 180 |
| Zone 3 | 180 |
| Zone 4 | 180 |
| Zone 5 | 170 |
| Zone 6 | 170 |
| Zone 7 | 170 |
| Zone 8 | 170 |
| Zone 9 | 160 |
| Die | 160 |

As a result of this example, it is observed that alkyl ether sulfates may experience thermal degradation during the compounding process due to high processing temperature. As an evidence of thermal degradation, the color of the extrudate may change from light yellow to dark brown. It also may not hold its structural integrity.

### Example 12

### Extrusion with Cationic Guar Polymer

In this example, a cationic guar polymer in powder form is introduced for the first time as a part of final composite. In order to investigate the effects of the cationic guar independently, the surfactant solution mixture of this example composite consists of only alcohol ethoxy sulfate and perfume. The contents of materials are exhibited in Table 16 below.

TABLE 16
Material Ingredients of Example 12

| Component | wt % |
|---|---|
| Polyvinyl alcohol | 28.19 |
| Glycerin | 14.09 |
| Alcohol ethoxy sulfate solution | 31.85 |
| (70% activity) | |
| Additional Water | 22.55 |
| Perfume | 1.97 |
| Jaguar C500 (cationic guar) | 1.35 |
| Overall Water | 32.10 |
| Polymer:Active Surfactant | 54.3:45.7 |
| Active alcohol ethoxy sulfate | 22.30 |

The cationic guar powder is weighed and blended with the pre-compounded PVOH/glycerin pellets. The pellet and powder mix is fed into the twin-screw extruder via using a gravimetric weight-loss feeder. The rest of processing conditions are maintained as aforementioned.

The bar samples may show a uniform and homogeneous mixing quality. The bar samples may not display the surface stickiness due to the absence of cocamidopropyl betaine and amino silicone fluid.

### Example 13

### Dissolving Solid Bar with Polyquaternium 76

For this example, similar to Example 12, polyquaternium 76 (Mirapol AT-1 from Rhodia Inc.) is first introduced to the final composite. This liquid type cationic polymer is weighed and mixed with the rest of surfactant solution mixture ingredients, which are alcohol ethoxy sulfate and perfume. The material contents of this example are shown in Table 17.

TABLE 17
Material Ingredients of Example 13

| Component | wt % |
|---|---|
| Polyvinyl alcohol | 28.50 |
| Glycerin | 14.25 |
| Alcohol ethoxy sulfate | 32.20 |
| (70% activity) | |
| Additional Water | 22.80 |
| Perfume | 1.99 |
| Polyquaternium 76-Mirapol AT-1 | 0.26 |
| (10% activity) | |
| Overall Water | 32.10 |
| Polymer:Active Surfactant | 55.8:44.2 |
| Active alcohol ethoxy sulfate | 22.54 |
| Active coacervate | 0.026 |

The samples of this example may not stimulate any surface stickiness because cocamidopropyl betaine and amino silicone fluid may not be employed in the final formulation.

### Example 14

### Extrusion with Polyquaternium 76, Cocamidopropyl Betaine, and Amino Silicone Fluid

In this example, a polyquaternium 76 (Mirapol AT-1 from Rhodia Inc.) is utilized with cocamidopropyl betaine and amino silicone fluid for the final composite formulation. The surfactant solution mixture of this example consists of alcohol ethoxy sulfate, cocamidopropyl betaine, perfume, and liquid type coacervate. The material contents of this example are listed in Table 18.

TABLE 18
Material Ingredients of Example 14

| Component | wt % |
|---|---|
| Polyvinyl alcohol | 27.38 |
| Glycerin | 13.69 |
| Alcohol ethoxy sulfate | 26.28 |
| (70% activity) | |
| Cocamidopropyl betaine | 4.64 |
| Additional Water | 21.90 |
| Perfume | 1.92 |
| Polyquaternium 76-Mirapol AT-1 | 0.22 |
| (10% activity) | |
| Amino Silicone Fluid | 3.97 |
| Overall Water | 33.03 |
| Polymer:Active Surfactant | 58.0:42.0 |
| Active alcohol ethoxy sulfate | 18.39 |
| Active cocamidopropyl betaine | 1.39 |
| Active coacervate | 0.022 |
| Active SLE1S:Active CAPB | 93.0:7.0 |

Amino silicone fluid is injected separately at the flange-pipe between the twin-screw extruder and the single-screw extruder and its injection flow rate is determined based on its weight ratio and the actual throughput of extrusion system when it is not injected.

For the bar samples of this example, they are cooled in the room temperature for more than 12 hours as the most of previous examples. Due to the combined effect of cocamidopropyl betaine and amino silicone fluid, however, the dissolving bar samples are still very sticky after the cooling phase and their shapes are deteriorated when they are forced to be taken out from the molds. In order to overcome this challenge, new method of cooling, which cools the bar samples inside of cold storage with the temperature range from −10 to −20° C. for two hours, is introduced. As a result, the samples are taken out from the molds with less amount of force and they are able to retain their original bar shapes. The samples are still stuck to the mold during this new cooling cycle although the degree of stickiness reduced significantly.

### Discussion of Examples

### Effects of Additional Water

As discussed in the above examples, additional water may be used as another plasticizer and a mixing enhancer. Lack of additional water may result in poor mixing between the PVOH/glycerin matrix and alkyl ether sulfate/perfume solution phase. When the additional water is not present, the PVOH/glycerin matrix may not be plasticized enough to be effectively mixed with the alkyl ether sulfate/perfume solution phase. Due to poor plasticization, the PVOH/glycerin matrix may form agglomerated solid particles within the surfactant solution phase. In Example 1, the solution mix already contains 13.47 wt % of water, but this water does not participate in the plasticization of PVOH/glycerin or the mixing according to the results of Example 1.

In the case of Example 2, the quality of final dissolving solid bar may be improved with using only 6.56 wt % of additional water. Since the additional water may act as a plasticizer for the PVOH/glycerin matrix, the viscosity of the PVOH/glycerin matrix may be reduced. The reduction of viscosity may yield better mixing behavior with the low viscous surfactant solution mix. In addition, the plasticization effect of additional water may lower the processing temperature range of the PVOH/glycerin matrix further. This may help to maintain the processing temperature lower than about 120° C. to prevent any thermal degradation of alkyl ether sulfate, which may occur during the extrusion process. Since both of the PVOH/glycerin matrix and the surfactant solution are water-soluble, the additional water may become a medium between these two main phases as well.

As it is described in the examples, increased content of additional water may improve the mixing behavior of the extrudate. However, the content of additional water may affect the quality and structural integrity of the extrudate significantly. Thus, the additional water content may be determined carefully to achieve desired hardness and performance levels of extruded personal care articles.

### Effects of Cocamidopropyl Betaine

Cocamidopropyl betaine (CAPB) may improve the dissolving behavior of the final product. However, it may yield negative traits on the final product such as stickiness on the product surface. When the content of CAPB increases, the stickiness of the final products may become more severe and it may present another challenge for the shaping stage of the manufacturing process. When the weight ratio between active alcohol ethoxy sulfate and active cocamidopropyl betaine is maintained as 93:7, the extrudate may not exhibit any signs of surface stickiness. The stickiness becomes more noticeable when the weight ratio becomes 87.5:12.5 and 82.6:17.4. Further, the increase of CAPB content may require a significant amount of additional water to accomplish good mixing between the PVOH/glycerin matrix and the surfactant solution phase. In one embodiment, CAPB content may be equal to or less than that of the SLE1S content.

### Effects of Injection Locations

A higher content of cocamidopropyl betaine may present more difficulty in obtaining a uniform phase of composite during the compounding process because the cocamidopropyl betaine may interact with the water predominantly and may prevent the water from actively participating in plasticizing PVOH. This may result in non-uniform mixing of the extrudate. As an attempt to overcome this difficulty and accomplish uniform mixing between PVOH and the solution mix, a high amount of water may be employed, but this may force a reduction in PVOH content. Consequently, this may result in negative characteristics such as reduced overall hardness and sticky surface. A more effective and convenient strategy may be exchanging the injection locations of the additional water and the solution mix. This technique may force the water to interact with PVOH prior to the cocamidopropyl betaine so it may successfully serve as a plasticizer for PVOH and a mixing enhancer between PVOH and the solution mix. Therefore, the desired overall hardness level may be accomplished without increasing the water content.

It may be realized that injection of water prior to injection of solution mix may enhance the mixing quality of the extrudate and may contribute positively to the properties of the final product. It may be suitable to inject the surfactant solution mix prior to the injection of additional water during the second extrusion compounding process when the content of cocamidopropyl betaine is low or when the surfactant solution mix consists of SLE1S and perfume. However, the additional water may be injected before the injection of the surfactant solution mix to minimize the additional water content and to achieve an adequate hardness for the extrudate.

### Effects of Processing Temperature

The restriction of processing temperature is thermal degradation of alcohol ethoxy sulfate. When the processing temperature is higher than the thermal degradation point of alcohol ethoxy sulfate, thermally degraded alcohol ethoxy sulfate may changeits color and produce a considerable amount of gas. When the gas is generated, the alcohol ethoxy sulfate solution may experience phase-separation and lose its viscosity significantly. At the end of this compounding process, consequently, the composite may not be able to maintain structural integrity, may become dark brown color, and may provide a very strong unpleasant odor. Therefore, it may be necessary to maintain the processing temperature to be equal to or lower than 120° C. from the injection location of surfactant solution mix to the end of the extrusion process.

### Effects of Surfactant Solution to Additional Water Weight Ratio

As the surfactant content increases, already cooled dissolving solid bar samples may begin to show the signs of material phase separation. In addition, the samples may exhibit the material phase separation once the personal care articles are cooled and stored more than few days in a room temperature environment. These samples may have a surfactant solution (i.e. alcohol ethoxy sulfate solution and cocamidopropyl betaine solution) to additional water ratio value of higher than 1.907. Materials without phase separation may have a surfactant solution to additional water weight ratio of about 1.413. As an effort to utilize the maximum amount of surfactant content without material phase separation, the ratio of 1.613 is attempted. This attempt still yields inconsistent results. In order to verify that 1.413 is the maximum ratio without material phase separation, the ratio of 1.255 is studied and the dissolving bar samples may not show any signs of material phase separation. Consequently, the ratio of surfactant solution to additional water contents is maintained lower than 1.413.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited herein are incorporated herein by reference in their entirety; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for forming a personal care article comprising:
a. producing an extrudate from a twin screw extruder; and
b. forming the extrudate into the personal care article, the personal care article comprising:
i. from about 10% to about 60% of one or more anionic surfactants, by weight of the personal care article, wherein the one or more anionic surfactants have a Krafft point of less than 30° C.;
ii. from about 10% to about 50% of one or more water soluble polymers, by weight of the personal care article;
iii. from about 1% to about 30% of one or more plasticizers, by weight of the personal care article; and
iv. from about 10% to about 50% water, by weight of the personal care article.

2. The process of claim 1, wherein the one or more anionic surfactants have a Krafft point of less than 25° C.

3. The process of claim 1, wherein the one or more anionic surfactants comprises one or more alkyl ether sulfates according to the following structure: [chemical structure]
wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
wherein M+ is a monovalent counterion selected from a group consisting of sodium, potassium, ammonium, protonated monoethanolamine, protonated diethanolamine, and protonated triethanolamine; and
wherein x is on average from about 0.5 moles to about 3 moles.

4. The process of claim 3, wherein x is on average from about 0.5 moles to about 3.0 moles of ethylene oxide.

5. The process of claim 3, wherein x is on average from about 1.0 mole to about 2.0 moles of ethylene oxide.

6. The process of claim 3, wherein the alkyl ether sulfate is sodium laureth sulfate.

7. The process of claim 1, wherein the personal care article comprises from about 15% to about 50% of one or more anionic surfactants, by weight of the personal care article.

8. The process of claim 1, wherein the personal care article comprises from about 20% to about 40% of the one or more anionic surfactants, by weight of the personal care article.

9. The process of claim 1, wherein the personal care article comprises from about 15% to about 45% water, by weight of the personal care article.

10. The process of claim 1, wherein the personal care article comprises from about 20% to about 40% water, by weight of the personal care article.

11. The process of claim 1, wherein the one or more water soluble polymers is selected from the group consisting of polyvinyl alcohol, polyvinylpyrrolidone, polyalkylene oxide, starch, starch derivatives, pullulan, gelatin, hydroxypropylmethylcellulose, methycellulose, carboxymethycellulose, and mixtures thereof.

12. The process of claim 1, wherein the one or more water soluble polymers is polyvinyl alcohol.

13. The process of claim 1, wherein the one or more plasticizers is selected from the group consisting of glycerin, propylene glycol, polyols, copolyols, polycarboxylic acids, polyesters, dimethicone copolyols, and mixtures thereof.

14. The process of claim 1, wherein the personal care article further comprises a secondary surfactant selected from the group consisting of amphoteric surfactants, zwitterionic surfactants, and mixtures thereof; and wherein the ratio of the one or more anionic surfactants to the secondary surfactant is from about 10:1 to about 1:2.

15. The process of claim 1, wherein the personal care article further comprises from about 0.1% to about 15% of one or more benefit agents.

16. The process of claim 15, wherein the one or more benefit agents are selected from the group consisting of anti-dandruff agents, conditioning agents, moisturizers, and combinations thereof.

17. The process of claim 16, wherein the conditioning agent is selected from the group consisting of silicones, aminosilicones, quaternized silicones, and combinations thereof.

18. The process of claim 1, wherein the personal care article further comprises a cationic polymer.

19. A process of forming a personal care article comprising:
a. adding one or more water soluble polymers and one or more plasticizers to a twin screw extruder to form a premix;
b. heating the premix to from about 150° C. to about 400° C.;
c. cooling the premix to below 135° C.;
d. mixing one or more anionic surfactants with the premix to form a mixture;
e. extruding the mixture from the twin screw extruder to produce an extrudate; and
f. forming the extrudate into the personal care article, the personal care article comprising:
i. from about 10% to about 60% of one or more anionic surfactants, by weight of the personal care article, wherein the one or more anionic surfactants have a Krafft point of less than 30° C.
ii. from about 10% to about 50% of one or more water soluble polymers, by weight of the personal care article;
iii. from about 1% to about 30% of one or more plasticizers, by weight of the personal care article; and
iv. from about 10% to about 50% water, by weight of the personal care article.

20. The process of claim 19, wherein the one or more anionic surfactants comprises an alkyl ether sulfate according to the following structure: [chemical structure]
wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
wherein M+ is a monovalent counterion selected from a group consisting of sodium, potassium, ammonium, protonated monoethanolamine, protonated diethanolamine, and protonated triethanolamine; and
wherein x is on average from about 0.5 moles to about 3 moles.
